(19) United States
(12) Patent Application Publication
(10) Pub. No.: US 2012/0164707 A1
(43) Pub. Date: Jun. 28, 2012

(54) Method for Producing Intracellular Microorganisms Using a Protozoan as a Host

(76) Inventor: Joelle De Coninck, Clenay (FR)

(21) Appl. No.: 13/382,605

(22) PCT Filed: Jul. 7, 2010
(86) PCT No.: PCT/EP2010/059737

§ 371 Date: Mar. 20, 2012

(30) Foreign Application Priority Data
Jul. 7, 2009 (FR) 09 54685

(51) Int. Cl. C12P 1/00; C12P 1/04; C12N 1/10

(52) U.S. Cl. 435/170; 435/258.1; 435/41

(57) ABSTRACT

The present invention relates to a method for producing intracellular microorganisms such as bacteria, parasitic protozoans or viruses, for example, characterised in that said method essentially comprises using a culture of at least one protozoan as a host cell in which said parasitic or non-parasitic intracellular microorganisms proliferate, and then recovering said intracellular microorganisms. The invention therefore proposes an alternative to selecting a mammal cell lineage for the in vitro culture of intracellular microorganisms. Said method preferably comprises at least the following steps: cultivating at least one protozoan strain in a suitable culture medium; then seeding the protozoan culture with at least one strain of an intracellular microorganism; followed by incubation, during which the intracellular microorganism develops in the cytoplasm of the protozoan until the latter lyses; and finally, recovering the produced intracellular microorganism.

### FIELD OF THE INVENTION

The present invention relates to a method for producing intracellular microorganisms such as bacteria, yeasts, protozoans or viruses, in which the host cell is a protozoan.

### BACKGROUND TO THE INVENTION

It is well known to produce pathogenic or non-pathogenic intracellular microorganisms by culturing said microorganisms inside eukaryotic cells.

In order to do this, animal or human cell lines are utilized in vitro in the laboratory or on an industrial scale. These cell lines can be adherent or non-adherent. By “adherent lines” is meant cells needing to adhere to a support for their development and by “non-adherent lines” cell lines cultured in suspension in their nutritive medium.

It is necessary to choose a cell line called “host cell” depending on the microorganism to be cultured.

This type of method for producing microorganisms has numerous drawbacks. The doubling time of the “host cells” is particularly long, of the order of 24 hours. Moreover, cell culture media are complex, expensive and mostly require the use of additives of animal origin, such as foetal calf serum for example, which is not recommended by the European Pharmacopoeia, and/or the addition of antibiotics. Furthermore, it is particularly difficult to extrapolate the culture of these “host cells” on an industrial scale for two essential reasons:
When the cells are adherent, culture is limited by the culture surface area as these cells need to be fixed to a specially treated support in order to multiply.
When the cells are non-adherent, they can multiply in suspension in a nutritive liquid but, in this case, culture is limited by the problems of oxygen transfer as protein-rich media foam very rapidly and at present no compatible anti-foaming agent is known.

### BRIEF DESCRIPTION OF THE INVENTION

One of the purposes of the invention is therefore to remedy these drawbacks by proposing as an alternative to the method for producing microorganisms by culturing adherent or non-adherent cells from mammals, a method for producing intracellular microorganisms such as bacteria, protozoans or viruses for example, which is simple in design, inexpensive and provides a high yield.

To this end, and according to the invention, a method is proposed for producing intracellular microorganisms, remarkable in that it essentially consists of using a culture of at least one protozoan as host cell in which these parasitic or non-parasitic intracellular microorganisms proliferate, then recovering said intracellular microorganisms.

According to a first variant of implementation of the method according to the invention, the latter comprises at least the following stages:
(a)—culture of at least one protozoan strain in an appropriate culture medium,
(b)—seeding of the culture of stage (a) with at least one strain of an intracellular microorganism,
(c)—incubation, preferably at a temperature comprised between 35 and 39° C., advantageously close to 37° C. (+/−1° C.), in the case of parasites or between 28° C. and 30° C. in the case of bacteria, during which, preferably over a duration comprised between 2 and 3 days, the intracellular microorganisms develop in the cytoplasm of the protozoans until the natural lysis of the latter occurs at the latest,
(d)—optional anticipated lysis of the protozoans induced preferably by mechanical and/or thermal and/or chemical stress—advantageously by pH and/or osmotic and/or enzymatic variation,
(e)—recovery of the intracellular microorganisms produced.

Preferably, stage (c) is carried out until the natural lysis of the parasitic protozoans is brought about by the intracellular microorganisms and in that it optionally comprises a stage (a₁) of synchronization of the seeded culture of stage (a) by subjecting it to moderate stress, preferably a physical and/or chemical stress, so as to standardize the level of development of the protozoans before being parasitized by the intracellular microorganisms and thus to allow culture, then a substantially synchronous natural lysis of said protozoans and subsequently a release, also substantially synchronous, of the intracellular microorganisms, which regulates the production of the latter.

According to a second variant of implementation of the method according to the invention, the latter comprises at least the following stages:
(a′)—culture of at least one protozoan strain in an appropriate culture medium,
(b′)—seeding of the culture of stage (a) with at least one strain of an intracellular microorganism,
(c′)—incubation, preferably at a temperature comprised between 35 and 39° C., advantageously close to 37° C. (+/−1° C.) in the case of parasites or between 28° C. and 30° C. in the case of bacteria, during which, preferably over a duration comprised between 2 and 3 days, the intracellular microorganisms develop in the cytoplasm of the protozoans without continuing as far as natural lysis of the latter,
(e′)—recovery of the protozoans parasitized by the intracellular microorganisms.

According to an essential characteristic of the method according to the invention, the protozoan is non-pathogenic and/or comprises at least one cytostome.

Preferably, said protozoan is chosen from the following list of species: Tetrahymena, Colpidium, Paramecium, amoebae, dinoflagellates, euglenae and combinations of protozoans belonging to all or some of these species, the non-pathogenic ciliated protozoan Tetrahymena thermophila B3 ATCC 30387 being particularly preferred.

Another subject of the invention relates to a medicament comprising protozoans in which the microorganisms have proliferated and which are collected on completion of the method according to the invention.

A last subject of the invention relates to a ready-to-use kit for implementing the method according to the invention. Said kit comprises:
protozoan host cells in living form (culture) and/or in regenerable form, preferably in lyophilized form and/or in dried form and/or in frozen form;
at least one culture medium for these protozoans, this medium being able to be in ready-to-use form or in the form which can be reconstituted using preferably liquid products;
optionally means for regenerating the protozoan host cells;
optionally means for reconstituting the culture medium;
optionally culture supports;
optionally instructions for use.

Other advantages and characteristics will become more apparent from the following description of several variants of implementation, given by way of non-limitative examples, of the method for culture of intracellular microorganisms according to the invention, with reference to the single FIGURE representing a comparative graph of the number of cells/ml as a function of time of Tetrahymena alone, of Bifidobacterium bifidum alone and of Bifidobacterium cultured inside Tetrahymena.

### DETAILED DESCRIPTION OF THE INVENTION

The method for producing intracellular microorganisms according to the invention consists of using a culture of ciliated or non-ciliated, pathogenic or non-pathogenic protozoans as host cells of one or more types of intracellular microorganisms capable of passing through the wall of the protozoans and multiplying in the cytoplasm of the latter. These microorganisms therefore behave as commensals or as parasites of the protozoans which serve them as culture medium.

The method according to the invention thus proposes an alternative to the choice of a cell line for the in vitro culture of intracellular microorganisms.

According to a first implementation embodiment of the invention, the process comprises at least the following stages:
(a)—culture of at least one protozoan strain in an appropriate culture medium,
(b)—seeding of the culture of stage (a) with at least one strain of an intracellular microorganism,
(c)—incubation, preferably at a temperature comprised between 35 and 39° C., advantageously close to 37° C. (+/−1° C.) in the case of parasites or between 28° C. and 30° C. in the case of bacteria, during which, preferably over a duration comprised between 2 and 3 days, the intracellular microorganisms develop in the cytoplasm of the protozoans until the natural lysis of the latter occurs at the latest,
(d)—optional anticipated lysis of the protozoans, preferably induced by mechanical and/or thermal and/or chemical stress—advantageously by pH and/or osmotic and/or enzymatic variation,
(e)—recovery of the intracellular microorganisms produced.

The lysis of the protozoans stops their growth and the growth of their commensal or parasitic microorganisms, and makes it possible to release the latter, optionally to separate them from the debris of lysed protozoans, then to collect them. These multiplied microorganisms can then be used in numerous applications.

Preferably, stage (c) is carried out until the natural lysis of the parasitic protozoans is brought about by the intracellular microorganisms.

Advantageously, this process with a stage (c) carried out until the natural lysis occurs, comprises a stage (a₁) of synchronization of the culture of the protozoan host before seeding (stage b) by subjecting it to moderate stress, preferably physical (temperature, centrifugation) and/or chemical stress, so as to standardize the level of development of the protozoans before they are parasitized by the intracellular microorganisms and thus allow culture then natural or anticipated and induced ([stage (d)] substantially synchronous lysis of said protozoans and subsequently also substantially synchronous release of the intracellular microorganisms, which regulates the production of the latter.

This moderate stress according to (a₁) is to be distinguished from the more severe stress used for the (complete) anticipated and induced lysis, which can be substituted for or added to the natural lysis occurring according to stage (c) of the first implementation embodiment. This moderate stress according to (a₁) does not lead to the lysis of the protozoans, but fixes the development of the protozoans which are in the most advanced stages of development and thus stops the development of all the protozoans at the same stage. The application of moderate stress to a culture of protozoans is a technique known to a person skilled in the art: LIOU, J J; FREDRICKSON, A G; SRIENC, F (1998). Selective synchronization of Tetrahymena pyriformis cell populations and cell growth kinetics during the cell cycle. Biotechnology Progress 14 Issue:3, 450-456.

This synchronization according to (a₁) makes it possible to release the intracellular microorganisms at the same moment and therefore to have a more regular production of the latter, which represents a certain asset for industrial production.

It is important that the anticipated, induced lysis of stage (d) of the first embodiment and a fortiori the moderate stress of synchronization stage (a₁) are aimed specifically at the protozoans and preserve the integrity of the intracellular microorganisms to be produced.

Thus, even if it is more drastic than the moderate stress utilized in synchronization stage (a₁), the stress of induced and anticipated lysis according to stage (d) advantageously remains tempered in particular when it is a thermal shock stress.

According to a second implementation embodiment of the invention, the method comprises at least the following stages:
(a′)—culture of at least one protozoan strain in an appropriate culture medium,
(b′)—seeding of the culture of stage (a) with at least one strain of an intracellular microorganism,
(c′)—incubation, preferably at a temperature comprised between 35 and 39° C., advantageously close to 37° C. (+/−1° C.) in the case of parasites or between 28° C. and 30° C. in the case of bacteria, during which, preferably over a duration comprised between 2 and 3 days, the intracellular microorganisms develop in the cytoplasm of the protozoans without continuing as far as natural lysis of the latter,
(e′)—recovery of the protozoans parasitized by the intracellular microorganisms.

Unlike the first implementation embodiment, the protozoans are not lysed and therefore constitute containers, or even transporters, for the commensal or parasitic microorganisms which have developed in their cytoplasms. Together these constitute new industrial products, for which numerous applications can also be envisaged.

In these two implementation embodiments, stages (a), (a′) of culture of the protozoans are carried out in an appropriate culture medium, such as the culture medium described in the publication DE CONINCK J., BOUQUELET S., DUMORTIER V., F. DUYME & VERDIER-DENANTES 1. (2000) Industrial media and fermentation processes for improved growth and protease production by Tetrahymena thermophila BIII Journal of Industrial Microbiology and Biotechnology, 24, 285-290. It is obvious that the protozoans can be cultured on any other appropriate culture medium well known to a person skilled in the art, without however exceeding the scope of the invention. Such culture media are generally based on yeast extract and glucose or skimmed milk. These are simple, inexpensive culture media, without the addition of foetal calf serum or antibiotics or other molecules.

Furthermore, culture of protozoans in a bioreactor, also called a fermenter or propagator, well known to a person skilled in the art, has been carried out successfully up to the 100 L scale and also according to the continuous technique with recirculation of the cells, described in the publication DE CONINCK J., BOUQUELET S., DUMORTIER V., F. DUYME & VERDIER-DENANTES 1. (2000) Industrial media and fermentation processes for improved growth and protease production by Tetrahymena thermophila BIII Journal of Industrial Microbiology and Biotechnology, 24, 285-290.

The cultures of the protozoans and of the commensals or parasites that they host according to the invention can also be carried out in a bioreactor.

After obtaining a suitable biomass of protozoans, the seeding according to stages (b) or (b′) of this culture with the intracellular microorganism for large-scale production, is carried out in an appropriate manner known per se. For example, an inoculum of the microorganism, in the form of liquid suspension, in the form of powder or in the form of colonies, is brought into contact with the culture of protozoans.

The incubation conditions in stages (c) or (c′) are chosen to optimize the growth of the microorganisms to be produced in the cytoplasm of these protozoans. The ideal temperature is generally between 35 and 39° C., advantageously close to 37° C. (+/−1° C.) in the case of parasites or between 28° C. and 30° C. in the case of bacteria. The duration depends on the protozoans and microorganisms to be produced, the temperature and the culture medium, but it is for example from 2 to 3 days.

Stages (e) and (e′) of recovery of the intracellular microorganisms produced, contained or not contained in their host protozoans are advantageously carried out by any method known to a person skilled in the art. Generally, the separation techniques used are decanting, centrifugation, filtration, or similar.

For stage (e) in which the culture medium comprises the lysates of protozoans, this recovery preferably comprises a filtration phase to remove the debris of the host cell.

According to the invention, it is possible to use one or more strains of protozoans and one or more strains of microorganisms to be multiplied.

Preferably, the choice of the protozoan(s) is made from the following list of species: Tetrahymena thermophila, Colpidium, Paramecium, amoebae, dinoflagellates, euglenae and combinations of protozoans belonging to all or some of these species; the non-pathogenic ciliated protozoan Tetrahymena thermophila B3 ATCC 30387 being particularly preferred. It will be noted that the Tetrahymena thermophila protozoans known for a long time known to be present in water constitute “reservoirs” or “sacs” for the bacteria Legionella pneumophila which are responsible for legionellosis, or other bacteria such as coliform bacteria. This is the case in particular in the scientific publications “KIKUHARA HIROYUKI et al.: “Intracellular multiplication of Legionella pneumophila in Tetrahymena thermophila” Journal of UOEH, vol. 16, no. 4, 1994, pages 263-275”, “AL-KHODOR SOUHAILA et al.: “The PmrA/PmrB two-component system of Legionella pneumophila is a global regulator required for intracellular replication within macrophages and protozoa” Infection and immunity, January 2009, vol. 77, no. 1, pages 374-386” and “STRAHL EILEEN D et al.: “Fluorescent acid-fast microscopy for measuring phagocytosis of Mycobacterium avium, M. intracellular e, and M. scrofulaceum by Tetrahymena pyriformis and their intracellular growth” Applied and Environmental Microbiology, October 2001, vol. 67, no. 10, pages 4432-4439”.

This protozoan Tetrahymena thermophila of strain B3 ATCC 30387 can cany out phagocytosis. Furthermore, it has a cytostome giving it an indisputable additional advantage over other strains of protozoans such as amoebae for example which only cany out phagocytosis. This advantage avoids searching for the optimum cell line/intracellular microorganism combination. The development of the intracellular microorganism takes place in the cytoplasm of the protozoan until lysis of the latter occurs, or until it is stopped by, for example moderate mechanical and/or thermal and/or chemical lysis—advantageously by pH and/or osmotic variation. Moreover, it will be observed that the protozoan Tetrahymena thermophila has the advantage of being a G.R.A.S (Generally Recognized As Safe) organism, providing an absence of pathogenicity to humans, which can be cultured on simple and inexpensive media, extrapolated to an industrial scale, with a particularly short generation time of approximately 2 hours.

In a general fashion, all the protozoans which can be the site of endocytosis as a method for particles to pass through the cell wall are potential candidates for the method according to the invention.

The commensal or parasitic intracellular microorganisms of the culture support protozoans can in particular be bacteria, parasitic or non-parasitic protozoans or viruses.

By way of examples of microorganisms capable of being produced by the method according to the invention, there may be mentioned: parasites belonging or not belonging to the apicomplex phylum, and viruses or bacteria particularly difficult to culture in vitro owing to the environmental conditions that they require, namely: Listeria, Legionella, Bifidobacterium.

As regards the applications of the method according to the invention, they correspond to all the cases where there is a need to rapidly produce microorganisms of interest in significant numbers and quality, and under economical conditions. This relates in particular to the health, food, dietetic, phytosanitary and cosmetic fields and the fields of industrial fermentation, industrial biotransformation (for example of geological raw materials) and biological purification.

In particular, the invention relates to a culture method as defined above characterized in that it is applied to the multiplication of an infectious agent or agents constituting the active ingredient or ingredients of vaccines, within the context of the manufacture of said vaccines.

The invention can thus relate to a method for manufacturing vaccines involving the culture method as defined above for the multiplication of the infectious agent or agents constituting the active ingredient or ingredients of vaccines.

Another subject of the invention is a culture method as defined above characterized in that it is applied to the culture of microorganisms for the production of metabolites of interest by fermentation.

Another subject of the invention is a culture method as defined above characterized in that the microorganisms produced are ferments.

Another subject of the invention is a culture method as defined above characterized in that it is applied to the culture of microorganisms of interest intended for industrial biotransformation or biological purification.

The invention can thus relate to an industrial biotransformation or biological purification method involving the culture method as defined above for the multiplication of the active ingredient or ingredients in industrial biotransformation or biological purification.

With respect to the second implementation embodiment of the culture method according to the invention, another subject of said invention is the use of the protozoans in which the microorganisms have proliferated and which are collected on completion of the culture method as defined above, as a dietetic food component (nutriceutical) or as an active ingredient in a phytosanitary or cosmetic composition.

Still with respect to the second implementation embodiment of the culture method according to the invention, another subject of said invention is a medicament comprising protozoans in which the microorganisms have proliferated and which are collected on completion of the culture method as defined above.

The invention finally relates to a “ready-to-use kit” for implementing the culture method as defined above characterized in that it comprises:
protozoan host cells in living form (culture) and/or in regenerable form, preferably in lyophilized form and/or in dried form and/or in frozen form;
at least one culture medium for these protozoans, this medium being able to be in ready-to-use form or in the form which can be reconstituted using preferably liquid products;
optionally means for regenerating the protozoan host cells;
optionally means for reconstituting the culture medium;
optionally culture supports;
optionally instructions for use.

### Example 1

By way of a first example, the production of an apicomplexan in its tachyzoite infectious form is described below.

The Apicomplexa phylum constitutes an ancient and diversified group of parasites comprising more than 4,000 species. They are responsible for diseases with a high economic and health impact in veterinary health (toxoplasmosis, neosporosis, cryptosporidiosis, coccidiosis, babesiosis, etc.) and also in human health (toxoplasmosis, malaria, babesiosis, etc.). Producing these apicomplexans is therefore of interest in basic research and also for the production of vaccines and other therapeutic forms.

Among these apicomplexans, the first example chosen is the production of Toxoplasma gondii, the toxoplasmosis agent, the production method of which is described in detail.

The first stage (a) consists of culturing the Tetrahymena thermophila strain B3 ATCC 30387 on one of the culture media described in the publication DE CONINCK J., BOUQUELET S., DUMORTIER V., F. DUYME & VERDIER-DENANTES 1. (2000) Industrial media and fermentation processes for improved growth and protease production by Tetrahymena thermophila BIII Journal of Industrial Microbiology and Biotechnology, 24, 285-290: a so-called YEG (yeast extract and glucose) medium and a so-called MYE (skimmed milk and yeast extract) medium.

The culture was carried out in stoppered test tubes (9 mL) at 28° C. over 2 days (non-optimized culture).

The quantity of protozoans produced under these non-aerated conditions on MYE is 1.8×10⁵/mL. It is recalled that in the presence of air, the quantity achieved in a fermenter is of the order of 2×10⁷/mL DE CONINCK J., BOUQUELET S., DUMORTIER V., F. DUYME & VERDIER-DENANTES 1. (2000) Industrial media and fermentation processes for improved growth and protease production by Tetrahymena thermophila BIII Journal of industrial Microbiology and Biotechnology, 24, 285-290).

In order to seed these produced protozoans (host cells), Toxoplasma gondii tachyzoites WTD ATCC 50779 are produced by culture on the HFF (Human Foreskin Fibroblast) cell line in 25 cm² flasks containing 5 mL of DMEM medium (Dulbecco's Modified Eagle's Medium) and 2% foetal calf serum. After 3 days of infection at 37° C., in the presence of 5% CO₂-enriched air, approximately 8×10⁶ tachyzoites/mL are produced in the culture supernatant.

A volume of 2 mL of the culture of Tetrahymena on the MYE medium described previously, was placed in a 25 cm² cell culture flask with a ventilated stopper. This culture was infected with 100 μL of Toxoplasma gondii tachyzoites freshly taken from an HFF cell-line culture after infection for 3 days.

The incubation temperature is 37° C.

After infection for 2 days, 100 μl of supernatant is removed and serves to seed 2 mL of “fresh” Tetrahymena: this is a first pass. In total, 7 infection passes were carried out successively, thus proving the viability and the infectiousness of the tachyzoites produced on the Tetrahymena strain.

Counts are carried out on part of the supernatant after filtration on a 5 μm filter in order to remove the cell debris and the tachyzoites produced were quantified: 2.5×10⁷/mL on average per pass. In order to verify the infectiousness of the tachyzoites produced, 100 μL of supernatant of tachyzoites removed from the Tetrahymena culture were added to 5 mL of DMEM medium containing HFF cells at 80% confluence. The lysis plaques were determined after only 2 days of infection, proving the infectious nature of the tachyzoites produced by the protozoan Tetrahymena.

The following summary table compares a culture of intracellular microorganisms using a ciliated protozoan as culture medium according to the invention with a culture of intracellular microorganisms from a mammal cell line according to the methods of the prior art.

| Culture from protozoans | Culture from mammal cells |
|---|---|
| GRAS organism (absence of | Animal or human cell lines which may |
| pathogenicity to humans) | be of cancerous origin |
| Maintenance of easy strains, similar to | Maintenance with far more precautions, |
| bacteria | with more elaborate techniques |
| | (trypsinization of adherent cells) |
| short generation time court ~2 hours | generation time of 24 hours |
| Possibility of achieving densities greater | population densities of the order of 10⁶ cells/ml |
| than 2 · 10⁷ cells/ml in only 24 hours | in 3 days on average |
| culture without serum | complex media based on serum of |
| simple media: the culture medium (for | animal origin |
| example PPYS) can be as much as one | |
| hundred times less expensive than those | |
| used for cultures of mammal cells | |
| can be easily cultured in an axenic | for adherent cells, limited culture |
| culture on a large scale, controlled, | defined by the surface area of the |
| simple and low-cost culture, making it | container, therefore limited number of |
| possible to produce a large number of | cells and complex and expensive |
| cells without being limited by the size of | techniques. |
| the container | For non-adherent cells, difficult to aerate |
| | and stir due to their fragility, which limits |
| | both the size and the performances of the |
| | industrial installations |
| Tolerates wide variations in osmotic | Fragility of the cells under |
| pressure (no osmolysis in distilled water) | environmental conditions |
| and temperature | |
| In a 25 cm² flask, on 2 mL of medium | In a 25 cm flask, on 5 mL of medium |
| containing Tetrahymena, i.e. 3.6 × 10⁵ | containing HFF cells, i.e. 5 × 10⁵ cells, 8 × |
| cells, 2.5 × 10⁷ tachyzoites/mL are | 10⁶ tachyzoites/mL are produced in 3-4 |
| produced in 2 days. | days. |

### Example 2

During the last decades the use of probiotic bacteria has attracted considerable attention as an accessible and risk-free means for the treatment of gastro-intestinal diseases. The bacteria which have been used for the treatment of diarrhea of viral or bacterial origin belong to the genera Lactobacillus and Bifidobacterium. The therapeutic activity of certain probiotic bacteria against gastroenteritis has been attributed to their ability to stabilize the mucosal barrier, produce anti-microbial substances, or also to stimulate intestinal immune defenses.

As a second example, and within the context of use as a probiotic, the production of an anaerobic bacterium Bifidobacterium bifidum, which is particularly difficult to culture, was carried out inside Tetrahymena thermophila by the method according to the invention.

In a first stage, Bifidobacterium bifidum ATCC 29 521 is cultured alone at 37° C. in MRS medium (Man, Rogosa, Sharpe, a medium suitable for the culture of lactic bacteria, supplemented with 0.5% L-cysteine HCL) over 24 hours. The population is of the order of 1.21×10⁶ cells/mL. The bacteria counts (in colony forming units (CFU)/mL) are carried out on MRS agar medium, supplemented with 0.5% cysteine HCl and incubation for 48 to 72 hours in an anaerobic jar. Then in a second stage, 1 mL of the culture of Bifidobacterium bifidum is added to a culture of the protozoan in 20 mL of YEG medium (see Example 1) obtained in 24 hours of stirred culture containing 2.35×10⁵ cells/mL. This co-culture is then carried out under aerobic conditions, stirred at 100 rpm, at 28° C. and in the YEG protozoan culture medium. In this medium, and under these culture conditions, the bacterium alone is incapable of developing.

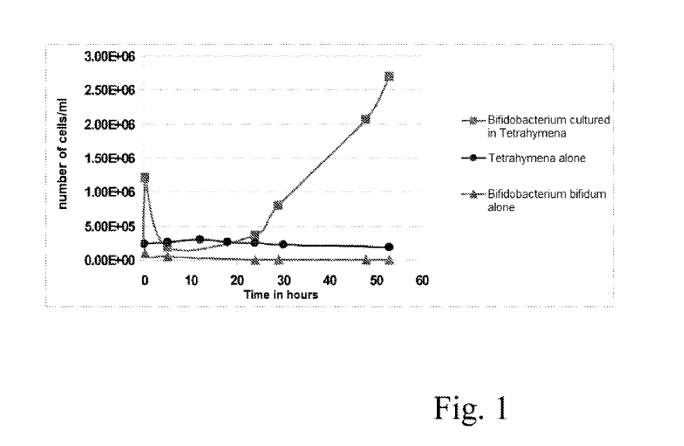

After culture for 24 hours, the quantity of Bifidobacterium bifidum has doubled inside Tetrahymena thermophila (see FIG. 1) while there are no more bacteria free in this medium. This quantity of bacteria inside the protozoan remains stable for 54 hours. In order to evaluate this bacterium inside the protozoan cells, 2 mL was removed then mechanically lysed through a syringe with a 25 G diameter needle in order to burst the Tetrahymena cells and thus release their contents (the lysis of the protozoan is estimated at approximately 80%). The bacteria count thus produced is carried out as described previously.

With reference to FIG. 1, it is clearly apparent that an anaerobic bacterium which is difficult to culture, in particular under aerobic conditions, can multiply inside a protozoan using methods dedicated to aerobic culture, i.e. which are easier to use and less expensive. If the probiotic bacterium multiplies and survives, this means that it has found a favourable environment inside Tetrahymena which protects it in particular from oxidative stress.

This example was carried out under non-optimized conditions and a method of improvement in order to exploit this co-culture can be to promote the multiplication of the protozoan during the co-culture in order to recover a greater number of Bifidobacterium bifidum on completion of the culture.

Finally, it is obvious that the examples which have just been given are only particular illustrations, in no event limitative with regard to the fields of application of the invention.

1. A method for producing intracellular microorganisms, such as an infectious agent or agents constituting the active ingredient or ingredients of vaccines, within the context of the manufacture of said vaccines, and/or of microorganisms for the production of metabolites of interest by fermentation, and/or of ferments and/or of microorganisms of interest intended for industrial biotransformation or biological purification and/or as a dietetic food component (nutriceutical) or as an active ingredient in a phytosanitary or cosmetic composition, wherein said method essentially consists of using a culture of at least one protozoan as host cell in which these parasitic or non-parasitic intracellular microorganisms proliferate, and recovering said intracellular microorganisms.

2. The method according to claim 1, which comprises at least the following stages:
(a) culture of at least one protozoan strain in an appropriate culture medium,
(b) seeding of the culture of stage (a) with at least one strain of an intracellular microorganism,
(c) incubation, preferably at a temperature comprised between 35 and 39° C., advantageously close to 37° C. (+/−1° C.) in the case of parasites or between 28° C. and 30° C. in the case of bacteria, during which, preferably over a duration comprised between 2 and 3 days, the intracellular microorganisms develop in the cytoplasm of the protozoans until the natural lysis of the latter occurs at the latest,
(d) optional anticipated lysis of the protozoans, preferably induced by mechanical and/or thermal and/or chemical stress—advantageously by pH and/or osmotic and/or enzymatic variation,
(e) recovery of the intracellular microorganisms produced.

3. The method according to claim 2, wherein stage (c) is carried out until the natural lysis of the parasitic protozoans is brought about by the intracellular microorganisms and said method optionally comprises a stage (a1) of synchronization of the seeded culture of stage (a) by subjecting it to moderate stress, preferably physical and/or chemical stress, so as to standardize the level of development of the protozoans before they are parasitized by the intracellular microorganisms and thus allow culture then substantially synchronous natural lysis of said protozoans and subsequently also substantially synchronous release of the intracellular microorganisms, which regulates the production of the latter.

4. The method according to claim 1, which comprises at least the following stages:
(a′) culturing at least one protozoan strain in an appropriate culture medium,
(b′) seeding the culture of stage (a) with at least one strain of an intracellular microorganism,
(c′) incubating, preferably at a temperature comprised between 35 and 39° C., advantageously close to 37° C. (+/−1° C.) in the case of parasites or between 28° C. and 30° C. in the case of bacteria, during which, preferably over a duration comprised between 2 and 3 days, the intracellular microorganisms develop in the cytoplasm of the protozoans without continuing as far as the natural lysis of the latter,
(e′) recovering the parasitic protozoans by the intracellular microorganisms.

5. The method according to claim 1, wherein the protozoan is non-pathogenic and/or comprises at least one cytostome.

6. The method according to claim 1, wherein the protozoan is chosen from the following list of species: Tetrahymena, Colpidium, Paramecium, amoebae, dinoflagellates, euglenae and combinations of protozoans belonging to all or some of these species; the non-pathogenic ciliated protozoan Tetrahymena thermophila B3 ATCC 30387 being particularly preferred.

7. A medicament comprising protozoans in which the microorganisms have proliferated and which are collected on completion of the method according to claim 4.

8. A Ready-to-use kit for implementation of the method according to claim 1, which comprises:
protozoan host cells in living form (culture) and/or in regenerable form, preferably in lyophilized form and/or in dried form and/or in frozen form;
at least one culture medium for these protozoans, this medium being able to be in ready-to-use form or in a form which can be reconstituted using preferably liquid products;
optionally means for regenerating the protozoan host cells;
optionally means for reconstituting the culture medium;
optionally culture supports;
optionally instructions for use.
